(19) United States
(12) Patent Application Publication
(10) Pub. No.: US 2010/0258123 A1
(43) Pub. Date: Oct. 14, 2010

(54) SYSTEMS AND /OR METHOD FOR CALIBRATION -LESS DEVICE OR LESS EXPENSIVE CALIBRATION DEVICES FOR TREATING SLEEP-DISORDERED BREATHING

(75) Inventors: Chinmayee Somaiya, Turramurra (AU); Steven Paul Farrugia, Lugarno (AU); Matthew Alder, Caboolture (AU); Kristian Thomsen, Drewvale (AU)

Correspondence Address: NIXON & VANDERHYE, PC, 901 NORTH GLEBE ROAD, 11TH FLOOR, ARLINGTON, VA, 22203, US

(73) Assignee: ResMed Limited, Belle Vista (AU)

(21) Appl. No.: 12/312,489

(22) PCT Filed: Jun. 5, 2007
(86) PCT No.: PCT/AU2007/000763

§ 371 Date: May 13, 2009

Related U.S. Application Data
(60) Provisional application No. 60/810,624, filed on Jun. 5, 2006.

(51) Int. Cl. A61M 16/00; A61M 16/06

(52) U.S. Cl. 128/204.23; 128/205.25

(57) ABSTRACT

Systems and/or methods for treating sleep-disordered breathing (SDB) are provided. In particular, systems and/or methods are provided that include software systems for use with auto-titrating devices (e.g. APAP devices) that reduce and/or eliminate the need to calibrate the auto-titrating devices. The software system also may reduce and/or eliminate the need for certain sensors used in such calibrations. Certain example embodiments compute snore based on noises measured during expiration and inspiration, and certain example embodiments set patient leak utilizing the vent flow level. Certain example embodiments change treatment pressure thresholds after measuring patient improvement by monitoring a variable correlated with actual delivery pressure in accordance with an example embodiment, and certain example embodiments provide pressure according to motor speed in accordance with an example embodiment.

### CROSS-REFERENCE TO RELATED APPLICATION

This application claims the benefit of U.S. Provisional Application No. 60/810,624, filed Jun. 5, 2006, incorporated herein by reference in its entirety.

### FIELD OF THE INVENTION

The example embodiments disclosed herein relate to systems and/or methods for treating sleep-disordered breathing (SDB). More particularly, the example embodiments disclosed herein relate to systems and/or methods that include software systems for use with auto-titrating devices that reduce and/or eliminate the need to calibrate the auto-titrating devices. The software system also may reduce and/or eliminate the need for certain sensors used in such calibrations.

### BACKGROUND OF THE INVENTION

Obstructive Sleep Apnea (OSA) and other dangerous sleep-disordered breathing (SDB) conditions affect thousands worldwide. Numerous techniques have emerged for the treating SDB, including, for example, the use of Continuous Positive Airway Pressure (CPAP) devices, which continuously provide pressurized air or other breathable gas to the entrance of a patient's airways via a patient interface (e.g. a mask) at a pressure elevated above atmospheric pressure, typically in the range 3-20 cm H₂O. Typically, patients suspected of suffering from an SDB register with a certified sleep laboratory where sleep technicians fit patients with numerous data collectors and monitor their sleep activity over a given period.

In an auto-titrating CPAP device, treatment parameters (e.g. pressure, flow, etc.) are measured at the blower end, while the output is the pressure delivered to the patient at the mask. Thus, the characteristics of the mask and the air delivery system must be known and accounted for to ensure correct treatment delivery. Specifically, to ensure proper treatment, a delivery device must compensate for any effects of the mask and/or the air delivery system on the delivered pressure. This is because auto-titrating devices typically have fixed responses to the severity of patient obstructive events, so the prescribed treatment pressure must be correctly translated to motor drive power. The compensation for the mask and/or the air delivery system achieves this objective.

Conventionally, to ensure proper treatment, extensive mask and airpath calibrations are performed. Typically, at the user-end, the user (and/or a clinician acting for the user) is required to provide the treatment device with details for all of the components of the patient interface system that are used. In most cases, the components of the patient interface system will comprise an array of elements, such as, for example, humidifier, antibacterial filter, air delivery tube, mask, etc. This process is cumbersome at the clinician level as well as at the production level because, for example, clinicians have to perform calibrations, while producers have to configure their treatment devices with sensors and other circuitry for use with the calibrations.

Thus, it will be appreciated that a need has developed in the art to overcome one or more of these and other disadvantages.

### SUMMARY OF THE INVENTION

One aspect of the invention relates to a positive airway pressure (PAP) system comprising a PAP device and a patient circuit comprising an air delivery conduit and a patient interface unit, wherein the PAP device is configured to deliver therapeutic treatment pressures requiring only a reduced or generic calibration of the system performed substantially independent of the specific patient circuit used. Optionally the device does not comprise either a pressure sensor or a flow sensor or both.

Certain example embodiments provide a method of delivering therapeutic treatment pressures to a patient via a positive airway pressure (PAP) device comprising an operable flow generator and a patient circuit including a patient interface unit. That method may comprise generically calibrating the device substantially independent of a specific patient circuit used; setting a first pressure; providing a supply of pressurized breathable gas to the patient at or close to the first pressure; monitoring a parameter indicative of a patient's condition over a period of time to measure patient improvement; and, when the monitored parameter indicates a lack of patient improvement, changing the first pressure.

Certain other example embodiments provide a system for delivering therapeutic treatment pressure to a patient suffering from sleep disordered breathing comprising a patient circuit operable to deliver the pressurized breathable gas to the patient; a controllable flow generator operable to generate a supply of pressurized breathable gas to be delivered to the patient at a first pressure substantially independent of the specific patient circuit used; a monitor operable to measure a parameter indicative of a patient's condition over a period of time; and, a processor operable to change the controllable flow generator's first pressure when the monitored parameter indicates a lack of patient improvement.

Further example embodiments provide a method of delivering therapeutic treatment pressure to a patient via a positive airway pressure (PAP) device comprising an operable flow generator and a patient circuit including a patient interface unit, with the method comprising generically calibrating the device substantially independent of the specific patient circuit used; setting a first pressure; providing a supply of pressurized breathable gas to the patient at or near the first pressure; monitoring a parameter indicative of a patient's condition over a period of time to measure patient improvement; and, when the monitored parameter indicates a lack of patient improvement changing the first pressure by adjusting an element in the PAP device to modify the amount of pressurized breathable gas provided to the patient.

Yet further example embodiments provide a system for delivering therapeutic treatment pressure to a patient suffering from sleep disordered breathing comprising a patient circuit operable to deliver the pressurized breathable gas to the patient; a controllable flow generator operable to generate a supply of pressurized breathable gas to be delivered to the patient at a first pressure substantially independent of the specific patient circuit used; a monitor operable to measure a parameter indicative of a patient's condition over a period of time; and a processor operable to change the controllable flow generator's first pressure and an element of the controllable flow generator; wherein the processor changes the first pressure and the element of the controllable flow generator when the monitored parameter indicates a lack of patient improvement.

Certain example embodiments provide a method of classifying mask leak for a patient using a positive airway pressure (PAP) device, that method comprising providing a supply of pressurized breathable gas to the patient at a first pressure; estimating vent flow based on the first pressure; determining the average value of flow; determining the mask leak based on the average value of flow and the estimated vent flow; and classifying the mask leak according to at least one predetermined mask leak threshold. Other example embodiments provide a method of treating a patient via a positive airway pressure (PAP) device, classifying mask leak using this method. Those example embodiments also may comprise monitoring at least one parameter indicative of a patient's condition over a period of time to measure patient improvement; and changing the PAP device first pressure, when the monitored parameter indicates a lack of patient improvement, and the mask leak is classified below at least one predetermined mask leak threshold.

Certain example embodiments provide a system for treating a patient suffering from sleep disordered breathing comprising a patient circuit configured to deliver pressurized breathable gas to the patient; a controllable flow generator operable to generate the pressurized breathable gas to be delivered to the patient at a first pressure independent of the specific patient circuit used; a processor configured to estimate the PAP device's vent flow based on the first pressure, determining the average value of flow, determining the mask leak based on the average value of flow and the estimated vent flow, and classifying the mask leak according to at least one predetermined mask leak threshold; and, a monitor operable to measure a parameter indicative of a patient's condition over a period of time; wherein the processor is operable to change the controllable flow generator's first pressure when the monitored parameter indicates a lack of patient improvement.

Still other example embodiments provide a method of treating a patient via a positive airway pressure (PAP) device, with the method comprising providing a supply of pressurized breathable gas to the patient at a first pressure; estimating vent flow based on the first pressure; determining the average value of flow; determining the mask leak based on the average value of flow and the estimated vent flow; classifying the mask leak according to at least one predetermined mask leak threshold; monitoring at least one parameter indicative of a patient's condition over a period of time to measure patient improvement; and, when the Monitored parameter indicates a lack of patient improvement, changing the PAP device first pressure.

Certain example embodiments provide a method of treating a patient suffering from sleep-disordered breathing. That method may comprise setting a first pressure; providing a supply of pressurized breathable gas to the patient at or close to the first pressure via a controllable flow generator; monitoring a parameter indicative of a patient's condition over a period of time to measure treatment efficacy; and, when the monitored parameter indicates a change in treatment efficacy, changing the first pressure. Optionally, the treatment's aggressiveness and/or gentleness may be adjusted based at least in part on the change in treatment efficacy.

Other aspects, features, and advantages of this invention will become apparent from the following detailed description when taken in conjunction with the accompanying drawings, which are a part of this disclosure and which illustrate, by way of example, principles of this invention.

### BRIEF DESCRIPTION OF THE DRAWINGS

The accompanying drawings facilitate an understanding of the various embodiments of this invention. In such drawings:

FIG. 1 is an exemplary flowchart showing a prior art process for using a CPAP device to treat a patient with SDB;

FIG. 1A is a detailed view of the calibrations conventionally required for CPAP treatment in the prior art;

FIG. 1B is a simplified partial schematic view of an auto-titration device connected to a patient for treatment in accordance with an example embodiment;

FIG. 2 is an exemplary flowchart showing a process for computing snore based on noises measured during expiration and inspiration in accordance with an example embodiment;

FIG. 3 is an exemplary flowchart showing a process for setting patient leak according to the vent flow and total average flow level in accordance with an example embodiment;

FIG. 4 is an exemplary flowchart showing a process for changing pressure thresholds after measuring patient improvement by monitoring a variable correlated with actual delivery pressure in accordance with an example embodiment; and,

FIG. 5 is an exemplary flowchart showing a process for providing pressure according to motor speed in accordance with an example embodiment.

### DETAILED DESCRIPTION OF ILLUSTRATED EMBODIMENTS

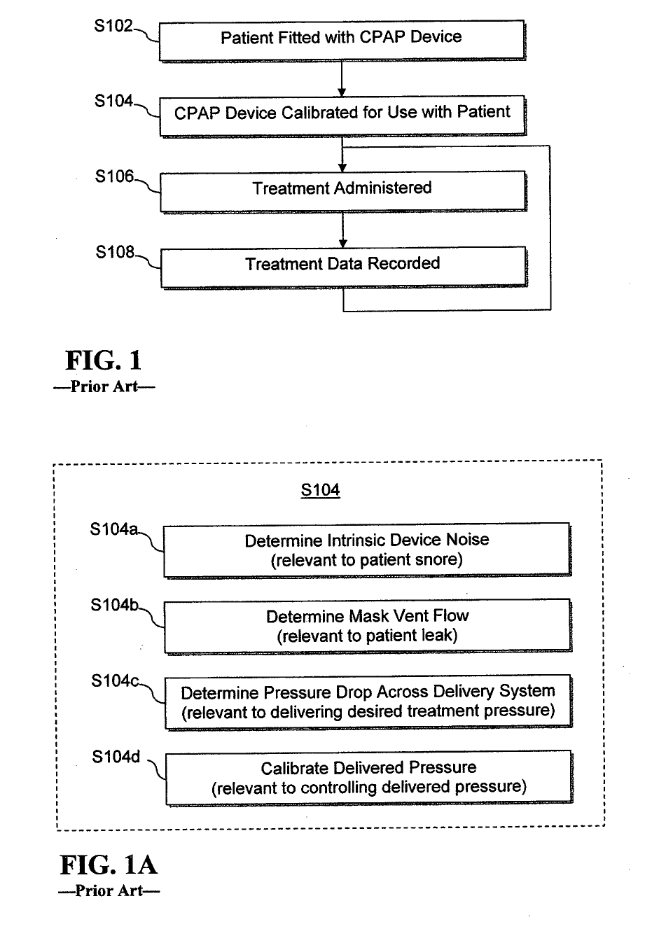

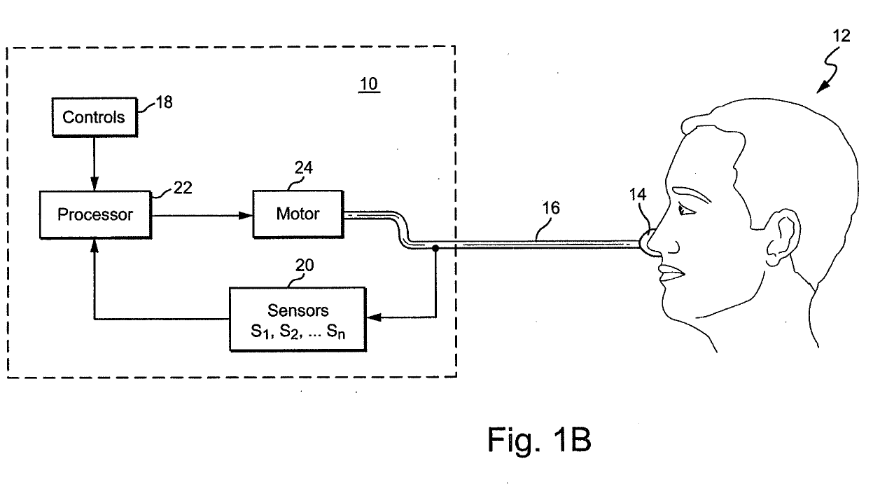

FIG. 1 is an exemplary flowchart showing a prior art process for using a CPAP device to treat a patient with SDB. In step S102, a patient is fitted with a CPAP device. That CPAP device is calibrated for use with the patient in step S104. Treatment is administered in step S106, and data treatment data is recorded in step S108. In some cases, after treatment data is recorded, the process may re-calibrate the CPAP device by returning to step S104 (not shown) before administering further treatment in step S106.

FIG. 1A is a detailed view of the calibrations conventionally required for CPAP treatment in the prior art. Essentially, FIG. 1A shows the calibrations comprising step S104 and pertaining to the above-described premises for CPAP treatment. Specifically, step S104a determines intrinsic device noise, which is relevant to determining patient snore. Step S104b determines mask vent flow, which is relevant to patient leak. Step S104c determines the pressure drop across the delivery system, which is relevant to delivering the desired treatment pressure. Step S104d calibrates delivered pressure, which is relevant to controlling the delivered pressure.

Existing solutions require a number of calibrations and rest on several premises common to CPAP devices. The first premise is that intrinsic device noise and patient snore are affected by the patient delivery system, and therefore the patient delivery system noise must be known in order to correctly estimate snore. The second premise is that the mask vent flow must be known in order to estimate patient leak, and the mask configuration therefore must be known. The third premise is that the pressure drop across the air delivery system must be known in order to deliver the desired treatment pressure, and the delivery tube elements therefore must be known. The fourth premise follows from the third. Specifically, the delivered pressure must be known in order to control it, therefore requiring calibration of pressure.

Certain example embodiments described herein can overcome one or more of the limitations presented by the above-described premises, thereby resulting in example devices that do not require patient calibration. Specifically, the premises do not apply as rigorously to Automatic Positive Airway Pressure (APAP) devices. Consequently, certain example embodiments may relax the above premises, balancing simplicity and precision, while still adequately fulfilling the requirements premises. Differently stated, certain example embodiments provide solutions that are simpler, though less precise, techniques for satisfying the above-described premises. Such embodiments can help reduce manufacturing and design costs, thereby making this technology available to patients at reduced costs, thereby helping improve patient care.

Such example systems are advantageous because, for example, they are less expensive to produce because they require fewer complicated sensors. Clinician also may benefit because, for example, such example systems are easier to set up because they require less (or no) calibration for the specific air delivery system used. Thus, such systems may also work with competitor masks and patient circuit elements.

Such an example system is shown in FIG. 1B, which is a simplified partial schematic view of an auto-titration device connected to a patient for treatment in accordance with an example embodiment. Auto-titration device 10 is connected to patient 12 for treatment. Patient 12 is fitted with mask 14, which provides pressurized breathable gas from auto-titration device 10 through flexible tube 16 directly to patient 12.

Auto-titration device 10 is comprised of several components. For example, an operator, sleep clinician, or patient can control various settings of auto-titration device 10 through controls 18. Controls 18 may allow control (e.g. manual control) of, for example, whether to begin treatment, duration of treatment, delivered pressure, etc. One or more sensors 20 monitor patient treatment information. As will be described below in greater detail, sensors 20 may help measure the information that enables the relaxation of the above described premises. For example, sensors 20 may include one or more of a noise sensor for detecting noises during inspiration and/or inspiration, a mask vent flow sensor, a pressure sensor, a patient leak sensor, sensor(s) for monitoring variables related to patient improvement, a motor speed detector, etc. It will be appreciated that these specific sensors may be implemented apart or in any combination, depending on the example embodiment implemented. Sensors 20 work with processor 22 to, for example, adjust treatment parameters, remove the need for some or all calibrations, etc. Processor 22 also controls motor 24 (along with other not pictured components) to control the supply of pressurized breathable gas. More detailed functionality of processor 22 will be described below.

### 1. Relaxation of Premises

1.1 The Patient Delivery System Must be Known to Correctly Estimate Snore.

The first premise is that intrinsic device noise and patient snore are affected by patient delivery system. Thus, conventionally, the patient delivery system must be known in order to correctly estimate snore. However, Application Ser. No. U.S. 60/756,709 filed on 6 Jan. 2006 entitled “Computer Controlled CPAP System with Snore Detection,” incorporated herein by reference in its entirety, is directed to techniques for detecting snoring in other ways. For example, snoring may be detected using the noise measured during expiration as the intrinsic device noise, and the additional noise measured during inspiration as snore. Thus, the treatment technique is independent of the patient circuit. Alternatively, this treatment technique can be conceived of as implicitly incorporating the characteristics of the patient circuit.

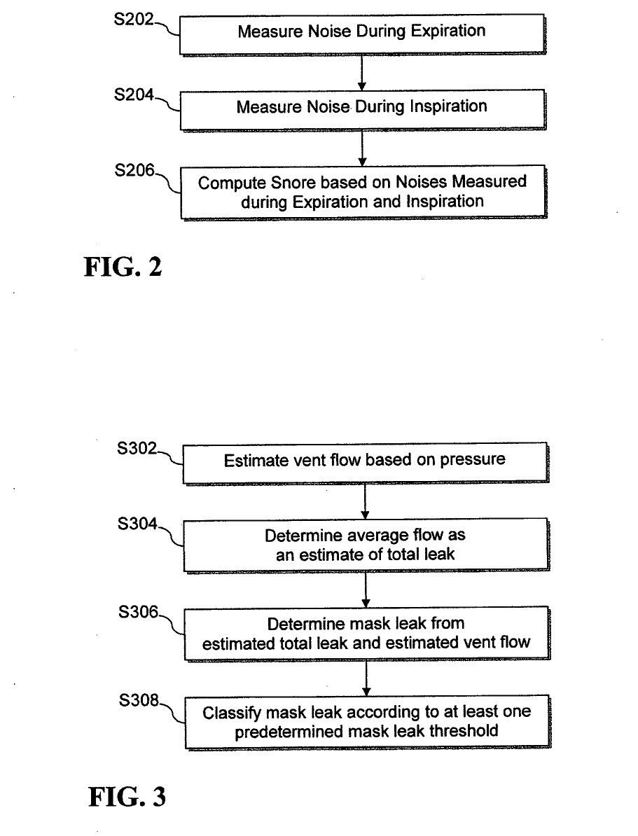

Thus, FIG. 2 is an exemplary flowchart showing a process for computing snore based on noises measured during expiration and inspiration in accordance with an example embodiment. Noise during expiration is measured in step S202, and noise during inspiration is measured in step S204. Step S206 may compute snore based on noises measured during expiration and inspiration (e.g. in steps S202 and S204, respectively) in the above-described manner.

Referring back to FIG. 1B, sensors 20 may perform the steps S202 and 5204 using one or more sensors, and processor 22 may compute the snore as in step S206 based on this information.

1.2 An Accurate Determination of Mask Vent Flow is Required to Estimate Patient Leak.

The second premise is that the mask vent flow must be known in order to estimate mask leak. However, in most cases, the exact amount of mask leak is not required. In fact, in most cases, only a rough estimate of mask leak is required to provide appropriate treatment. Accordingly, a rough estimate of vent flow provides a sufficiently accurate and reliable determination of patient leak. As such, certain example embodiments may only require a rough estimate of vent flow to derive a binary estimate of patient leak—for example, “high” or “low” leak. It will be appreciated that certain example embodiments may use a finer gradation by introducing additional levels of granularity (e.g. “high,” “medium,” or “low” leak).

Thus, FIG. 3 is an exemplary flowchart showing a process for setting patient leak according to the vent flow and total average flow level in accordance with an example embodiment. A rough estimate of vent flow is captured in step S302. The process may involve using a pressure to vent flow look-up table characteristic of an average mask as in step S304. The vent flow is therefore obtained from this table as the pressure is captured. Patient leak is then calculated as the average value of flow (which may be directly measured or estimated) minus the vent flow in step S306. This measure of leak is then graded according to pre-determined clinically valid thresholds. For example, leak above 0.4 l/sec is generally considered a high leak that requires intervention. This gradation result is thus associated with a set of discrete leak levels. Then, step S308 classifies the mask leak according to at least one predetermined mask leak threshold. The reason to measure or estimate the mask leak is to ensure that the treatment pressure is not increased when mask leak is high. If mask leak is high, then the treatment being delivered to the patient is not as effective. (Further, under these conditions, there may be loss of resolution and/or accuracy in treatment parameters). For example, despite the detection of respiratory events such as snoring or flow flattening the treatment pressure may not be increased. Increasing the treatment pressure may result in further increases in mask leak rather than providing more effective treatment and in some cases wake the patient. In general, the level of mask leak is logged and reported to notify the clinician that the system needs to be adjusted. For example, a different patient interface system may be required. Consequently the measure of mask leak is important to prevent increasing the treatment pressure in the presence of high leak. While it is important to prevent increasing the treatment pressure in the presence of high leak, it still may be advantageous to change the pressure thresholds after the leak reduces in certain example embodiments if a treatment efficacy indicator requires such a change.

Referring back to FIG. 1B, sensors 20 may perform the steps S302 (e.g. capture a rough estimate of vent flow) using one or more sensors, and processor 22 may classify the vent flow and set the patient leak based on this information.

1.3 The Pressure Drop Across the Air Delivery System Must be Known to Deliver the Desired Treatment Pressure.

The third premise is that the pressure drop across the air delivery system must be known in order to deliver the desired treatment pressure. While this premise generally holds for fixed CPAP devices, it may not be as critical for APAP devices. To a certain degree, pressure continues increasing until the patient airway condition improves. However, the threshold for treating a patient becomes more and more “severe” as the treatment pressure increases. For example, in other words, the patient is required to have increasingly worse episodes in order to be treated, as the treatment pressure increases. This is done to counter possible pressure runaway. The net result of this process is that the treatment saturates earlier than it would have, had there been a correct measure of “mask pressure.” Saturation implies that a stage is reached via the aforementioned mechanism where the threshold for treatment is unachievable by the patient, and therefore the patient now will not be treated even though he continues to experience obstructive events. For example, at 4 cm pressure snore equivalent of 60 dBA may be treated, whereas at 10 cm pressure, the snore may need to be 70 dBA to be treated.

A way to circumvent this problem in accordance with certain example embodiments is to change the threshold based on the improvement, or lack thereof, observed in the patient. There are several ways in which this process may be implemented:
Monitor the flow limitation over a period of time. If the flow limitation is consistently below a certain threshold, change the pressure increment for treatment of flow limitation.
Monitor the hourly Apnea-Hypopnea Index (AHI). If the hourly AHI does not drop below a given value (e.g. less than 2), change the threshold for treatment of apnea.
Monitor the arousal index. If the arousal index does not fall, change the threshold for one or more treatment algorithms.
   Thus, the delivered pressure in an APAP device may be altered without determining the actual delivery pressure.

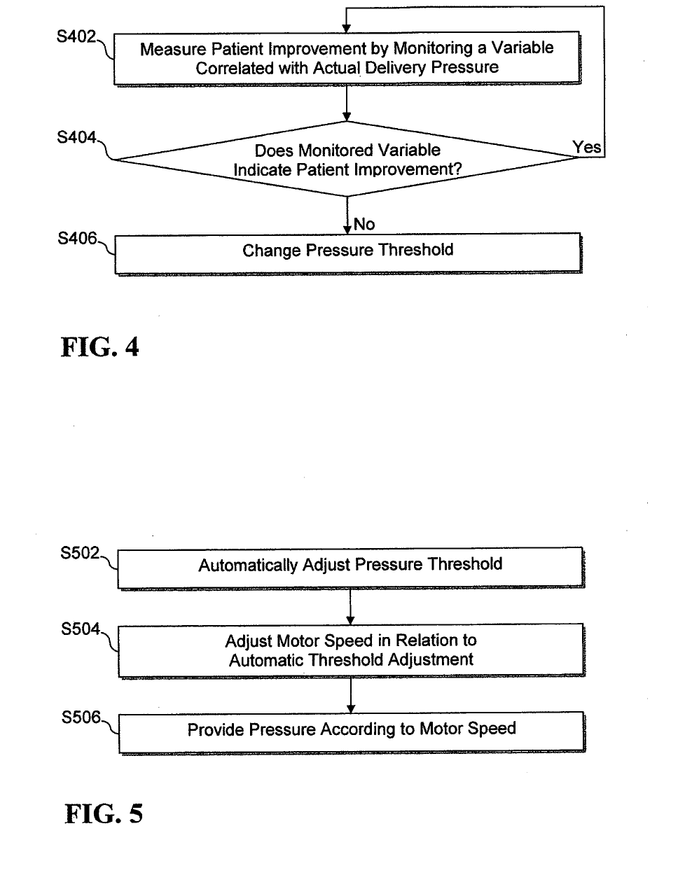

FIG. 4 is an exemplary flowchart showing a process for changing pressure after measuring patient improvement by monitoring a variable indicative of a patient's condition in accordance with an example embodiment. Step S402 measures patient improvement by monitoring a variable correlated with actual delivery pressure. For example, as described above, such variables may include flow limitation, hourly AHI, and/or arousal index. Step S404 determines whether the monitored variable indicates patient improvement. If it does, the process returns to step S402. However, if it does not indicate improvement, the pressure threshold is changed in step S406, allowing the treatment pressure to change. The process may then return to step S402 (not shown) to continue monitoring patient improvement during the course of treatment.

Referring back to FIG. 1B, sensors 20 may monitor one or more of flow limitation, AHI, and/or arousal index using one or more sensors. Processor 22 may determine whether a patient is improving and adjust the pressure based on this information.

1.4 The Delivered Pressure Must be Known to Control It, and Thus Pressure Must be Calibrated.

The fourth premise follows from the third. Specifically, the delivered pressure must be known in order to control it, therefore requiring calibration of pressure. However, Application Serial No. PCT/AU2005/001688 filed on 2 Nov. 2005 entitled “Using Motor Speed in a PAP Device to Estimate Flow,” incorporated herein by reference in its entirety, discloses techniques where delivered pressure is indirectly controlled. For example, delivered pressure may be indirectly controlled by controlling motor speed. Combining this technique with automatic threshold adjustment implies that the correct treatment pressure will be achieved without explicitly knowing what pressure is being delivered. Therefore, pressure calibration is not required.

Thus, FIG. 5 is an exemplary flowchart showing a process for providing pressure according to motor speed in accordance with an example embodiment. Step S502 automatically adjusts the pressure. Preferably, this may be accomplished by the process described with reference to FIG. 4. An element of the auto-titrating device (preferably the motor, and, more particularly, the motor's speed) is adjusted in relation to the automatic threshold adjustment in step S504. Pressure according to the element (e.g. motor speed) therefore can be provided in step S506. This process may continue to report pressure as the pressure automatically adjusts.

Referring back to FIG. 1B, processor 22 may monitor the automatic adjustments of pressure. When necessary, processor 22 further may adjust an element (e.g. motor 24) of auto-titrating device 10 to control the pressure of breathable gas supplied patient 12.

### 2. Example Systems

The concept of estimating mask leak and vent flow, for example, may be based on reducing the need to pre-calibrate all the different types of patient interface devices into the PAP device which leads to problems in backwards compatibility. Also, this requires the specific mask characteristics to be entered into the device when the device is set up. One concept is to estimate a generic set of mask characteristics that are programmed into the PAP device, which alleviates this calibration. The characteristics of the therapy may be monitored and ratio- and/or comparison-based assessments may be used as opposed to using absolute values. Another concept relates to providing a reduced and/or limited precalibration of the PAP device. For example it may be possible to specify which of several different types of patient interfaces are being implemented. For example, it may be possible to select full face mask, nasal mask, nasal prongs, etc., rather than having to select from a complete list of masks. In other example embodiments, neither generic calibration nor limited pre-calibration are necessary.

An example embodiment of a device capable of auto-calibration now will be described. It will be appreciated that the example embodiments described below, and the values and ranges discussed in connection therewith, are provided for illustrative purposes only and are not intended to limit the present invention.

2.1 Estimating Mask Pressure, and Regulating Mask Pressure by Speed Control

Following the relaxed premises above, air pressure increase over the ambient air pressure may be estimated at the flow generator so that mask pressure can be regulated. Mask pressure then may be regulated indirectly via speed control.

In an example embodiment, a flow generator is capable of sustaining patient mask air pressures ranging from approximately 5-20 cmH₂O at air flow rates of −90-180 liters/minute. It will be appreciated that to achieve the 20 cmH₂O top of the range, the demand will need to extend above 20 cmH₂O. Accuracy of the delivered pressure may be measured within ±0.5 cmH₂O+4% of the measured reading, assuming that the flow rate is approximately −30 to +120 liters/minute. The resolution of the set delivered mask pressure preferably is ≦0.2 cmH₂O, assuming a flow rate of approximate −30 to 120 liters/minute. Similarly, accuracy of the reported pressure may be measured within ±0.5 cmH₂O+4% of the measured reading, assuming that the flow rate is approximately −30 to +120 liters/minute.

Swings are measured with a manometer averaged over a number of sinusoidal breaths (e.g. 12 sinusoidal breaths). The swing target performance is ≦1.5 cmH₂O where the pressure is ≦10 cmH₂O, whereas the swing target performance is ≦2.0 cmH₂O at 10-20 cmH₂O. It will be appreciated that these figures represent the target performance for 15 breaths per minute at 500 ml tidal volume. Swings preferably are measured as out of phase swings, which may correspond to the reduction of pressure during inspiration, and vice versa. It will be appreciated, however, that swings may be measured as in-phase swings in certain example embodiments. One or more of sensors 20 may be configured to function as a manometer.

Jitter is the amplitude of pressure perturbations, measured at the mask with a water manometer, when the device is operated at a fixed pressure while connected to a blanked mask. Jitter preferably is <2 mmH₂O pp. This assumes that the jitter is measuring the mask pressure only and that there are no, or substantially no, leaks. One or more of sensors 20 may be configured to measure jitter.

The overall flow measurement accuracy preferably is ±12 liters/minute, assuming some respiratory flow. It will be appreciated that to achieve the required pressure accuracies, pressure feedback may be implemented (e.g. as controlled by processor 22 through motor 24 after readings are taken from sensors 20).

2.2 Capturing Data

Sensors 20 may capture data, and processor 22 may interpret this data. Preferably, certain data will be logged. Parameters may be logged every second, every breath, after every respiratory event, in real-time or at a specific sampling rate approximating real-time (e.g. 80 ms). One or more of the following parameters may be logged: motor speed, set pressure, mask pressure, mask leak, patient leak, flattening snore index, AHI, breath duration, event type, event duration, event time, and tidal volume. It will be appreciated that this list of parameters is for illustrative, non-limiting purposes only. Other parameters also may be captured along with, or in place of, one or more of the listed parameters.

2.3 Detecting Autoset Parameters

The following autoset parameters may be detected. It will be appreciated that these parameters are given for illustrative purposes only, and that they are not intended to limit the scope of the invention. Other parameters may be detected in addition to, or in place of, one or more of the below parameters.

2.3.1 Snore Detection

The snore detector may be implemented as a binary detector of inspiratory snore (e.g. one or more of sensors 20 may detect the presence or absence of a snore). A snore index may be computed (e.g. by processor 22) as the 5-breath moving average of the snore detector. The snore detector may detect snore in the range 0.0 to 2.0 “snore units” having a bandpass from about 30 to 100-300 Hz. This assumes a breath rate from approximately 6-30 bpm; a leak of approximately 0 to 1 liters/second; a minute volume of approximately 3-15 liters/minute; and a pressure range of approximately 5-20 cmH₂0.

2.3.2 Flow Limitation Detection

The flattening index (FI) may be computed (e.g. by processor 22) as a continuous variable, typically in the range of 0 to 0.34. More particularly, the FI is the 5 breath moving average of the FI calculated for the most recent 5 breaths, for example, at a resolution of 0.01 units. Typical values of the FI for ideal inputs are 0.0 for a square wave and 0.3 for a sine wave. A physiologically “normal breath” will have a value of approximately 0.25.

Linear combinations of sine and square wave inputs (e.g. from one or more of sensors 20) will produce an output that is equal to the sum of the outputs of the individual input waveforms. In response to the same input waveforms, the output of the flow limitation detector (e.g. as derived by processor 22) will be linearly correlated with the output of the autoset device. This assumes a breath rate of approximately 6-30 bpm; a leak of approximately 0-1 liters/second; a minute volume of approximately 3-15 liters/minute; and a Pressure range of approximately 5-20 cmH₂0.

Table 1 summarizes typical attributes, requirements, and underlying conditions relevant to the flattening index.

TABLE 1

| Attribute | Requirement | Condition |
|---|---|---|
| Range | 0.0-0.4 | |
| Accuracy | ±0.1 | Pressure range - 4-20 cmH₂O; |
| | | Minute volume 3-15 l/min; |
| | | Breath rate 6-30 bpm; |
| Expected values | Square wave 0.0 | Leak < 0.7 l/s |
| | Sine wave >0.3 | |
| | Normal breath | |
| | >0.24 | |
| Acceptable Error | | |
| Rate | | |
| False positive rate | 0% | Leak < 0.7 l/s |
| (e.g. flattening index | | |
| <0.19 for sine wave | | |
| breathing) | | |
| False negative rate | 0% | Leak < 0.7 l/s |
| (e.g. flattening index | | |
| >0.19 for square | | |
| wave breathing | | |
| Resolution | 0.01 | |

2.3.3 Apnea Detection

The apnea detector may detect (e.g. by one or more of sensors 20) the occurrence and duration of an apnea when there is a reduction in the measured ventilation to less than 25% of the long-term ventilation for duration of more than 10 seconds. The accuracy is about ±4 seconds or 20%, whichever is greater. The resolution is approximately 1.0 seconds. This assumes a breath rate of approximately 6-30 bpm; a leak of approximately 0-1 liter/second; minute volume of approximately 3-15 liters/minute; and a pressure range of approximately 5-20 cmH₂O. It will be appreciated that in some example embodiments, this detection is applicable after five minutes of steady breathing, and that there must be at least one minute between apneas for the above detection.

2.3.4 Hypopnea Detection

The hypopnea detector preferably will detect the occurrence of a hypopnea (e.g. through one or more of sensors 20) when there is a reduction in the measure of ventilation of more than 50% for duration of more than 15 seconds (e.g. as calculated by processor 22). The range of hypopnea detection is approximately >10 seconds, with an accuracy of approximately ±4 seconds, at a resolution of approximately 1.0 seconds. It will be appreciated that in certain example embodiments this detection becomes applicable after five minutes of steady breathing. This assumes a breath rate of approximately 6-30 bpm; a leak of 0-1 liters/second; a minute volume of approximately <15 liters/minute; and a pressure range of approximately 5-20 cmH20.

2.4 Detecting Other Device Parameters

The following device parameters may be detected (e.g. through one or more of sensors 20). It will be appreciated that these parameters are given for illustrative purposes only, and that they are not intended to limit the scope of the invention. Other parameters may be detected in addition to, or in place of, one or more of the below parameters.

2.4.1 Leak Measurement

Certain example embodiments may provide a broad quantitative indication of leak, to be used primarily for the detection of high leak. This indication may include both mouth (e.g. from patient 12) and mask leak (e.g. from mask 14). Table 2 summarizes typical attributes, requirements, and underlying conditions relevant to leak measurement. This assumes a breath rate of approximately 6-30 bpm; a leak of 0-1 liters/second; a minute volume of approximately <15 liters/minute; and a pressure range of approximately 4-20 cmH₂0.

TABLE 2

| Attribute | Requirement | Condition |
|---|---|---|
| Range | 0-120 l/min | |
| Accuracy | worst: ±20 l/min or ±30%, | Leak 0-60 l/min |
| | whichever is greater | |
| | preferred: ±10 l/min or ±20%, | Leak 60-120 l/min |
| | whichever is greater | |
| | monotonic | |
| Resolution | ±6 l/min | Leak 0-60 l/min |
| Bandwidth | 10 s time constant single | |
| | pole low-pass filter | |

2.4.2 Flow Estimation

Flow may be estimated using motor current (e.g. from motor 24). Table 3 summarizes typical attributes, requirements, and underlying conditions relevant to flow estimation.

TABLE 3

| Parameter | Specification |
|---|---|
| Range | −30 to 120 l/min |
| Respiratory Flow range | −60 to +60 l/min |
| Resolution | 1.2 litre/minute |
| Bandwidth | 7 Hz |

2.4.3 Auto-titrating of the CPAP Pressure

The flow generator may incorporate the following algorithms that allow it to auto-titrate the therapeutic CPAP pressure based on the detection of flow limitation (flattening), snoring, and apnea. In certain example embodiments, these algorithms may be implemented by processor 22 based on inputs from one or more of sensors 20. Similarly, in certain example embodiments, processor 22 may trigger certain responses (e.g. changing the speed of motor 24, altering pressure thresholds, etc.) based on data received from one or more of sensors 20 (e.g. indicating lack of patient improvement, etc.).

2.4.3.1 Response to Flattening

The flattening index is calculated over the last five breaths (e.g. by processor 22). If the index is less than a threshold value, the set pressure is increased by 3.0 cmH₂O for each unit by which the flattening index is less than the threshold. The default threshold is 0.22. The index may be recalculated each breath. The pressure increase (e.g. controlled by motor 24) due to flattening should be limited to a maximum of 1 cmH₂O per second.

2.4.3.2 Response to Snore

If the snore index is greater than a threshold value (default 0.2), the set pressure will be increased by 1.5 cmH₂O for each unit by which the snore is more than the threshold. The snore index will be recalculated (e.g. by processor 22) each breath. The pressure increase will be limited to a rate of 0.2 cmH₂O per second (i.e. 12 cm/minute). Table 4 indicates the response range for snore events of different durations.

TABLE 4

| | Expected treatment |
|---|---|
| | pressure after |
| | 8 normal breaths |
| Condition | (cmH₂O) |
| 4 cmH₂O, 500 ml, 15 bpm, 4 snore breaths | 5.0 to 8.8 |
| 4 cmH₂O, 500 ml, 15 bpm, 8 snore breaths | 6.0 to 12.0 |
| 4 cmH₂O, 500 ml, 15 bpm, 12 snore breaths | 8.0 to 14.0 |
| 4 cmH₂O, 500 ml, 30 bpm, 4 snore breaths | 5.0 to 8.0 |
| 4 cmH₂O, 500 ml, 30 bpm, 8 snore breaths | 5.5 to 10.0 |
| 4 cmH₂O, 500 ml, 30 bpm, 12 snore breaths | 5.8 to 12.6 |

2.4.3.3 Response to Apnea

The device incorporates the A10 algorithm in response to apnea. The A10 algorithm relates to a treatment algorithm where high pressure apneas are classified as central apneas, as taught in PCT Application No. WO 1999/24099, incorporated herein by reference in its entirety. U.S. Pat. Nos. 6,367,474, 6,502,572, 6,817,361, and 6,988,498 and U.S. Application No. 2006/0021618 also relate to the A10 algorithm, and each is incorporated herein by reference in its entirety. The A10 algorithm increases the APAP pressure, once the apnea is cleared, by an amount proportional to the apnea duration. The increment is limited such that the APAP pressure cannot exceed 10 cmH₂O in response to apneas. However, it will be appreciated that the APAP pressure may exceed 10 cmH₂0 in response to other physiological events (for example, snore). In certain example embodiments, these algorithms may be implemented by processor 22.

As an alternative to the A10 algorithm, the device may employ a closed-airway detection algorithm which can differentiate between open (i.e. central) and closed (i.e. obstructive) apneas. For example, if a central apnea is detected, the treatment pressure will not be increased. Examples of suitable closed-airway detection algorithms are described in U.S. Application Ser. No. 60/823,973 filed on 30 Aug. 2006 and U.S. Application Ser. No. 60/916,147 filed on 4 May 2007, each of which is incorporated herein by reference in its entirety.

Certain example embodiments preferably wait a required settling time at the minimum set pressure before responding to respiratory abnormalities. One example settling time is 5 minutes. A minimum settling time of 1 minute sometimes is advisable to allow the autoset algorithms to stabilize.

2.4.3.4 Response to Absence of Abnormalities

In the absence of abnormalities (e.g. detected apnea, hypopnea, snore, or flattening), the combined pressure may be reduced in increments exponentially towards their minimum, for example, with a 20-minute time constant.

It will be appreciated that the above-described techniques can be used to monitor treatment efficacy. Such monitored data may be used with or without a PAP device. In the former case, data regarding the patient's condition simply may be reported to a treating physician, sleep lab technician, etc. In the latter case, pressure can be adjusted based on the treatment efficacy. Thus, the treatment may be patient-based rather than device-based.

It also will be appreciated that the aggresiveness and/or gentleness of the treatment may be changed based on a measurement of treatment efficacy. For example, under normal conditions, pressure may be increased by 2 cm H₂O/10 dB snore/breath. A parameter may indicate a lack of efficacy (e.g. snore may not be reduced appropriately), and the treatment may accordingly change to 3 cm H₂O/10 dB snore/breath. Conversely, snore may be reduced more quickly than expected. In such cases, treatment may be reduced to 1 cm H₂O/10 dB snore/breath.

It will be appreciated that automatic calibration systems and/or learning systems may be used in connection with the above-described embodiments, including, for example, using acoustic ping to generate acoustic pictures to characterize the system. For example, learning circuits, connector recognition, smart mask systems, and/or tracking systems may be used in connection with the example embodiments described above. Such techniques are taught, for example, by U.S. application Ser. No. 10/450,519 filed on Nov. 6, 2003, U.S. application Ser. No. 10/637,771 filed on Aug. 8, 2003, U.S. Application Ser. No. 60/823,934 filed on Aug. 30, 2006, Application Serial No. PCT/AU2006/000679 filed on May 22, 2006, Application Serial No. PCT/AU2006/000238 filed on Feb. 24, 2006, and U.S. application Ser. No. 11/642,963 filed on Dec. 21, 2006, the entire contents of each of which are incorporated herein by reference.

While the invention has been described in connection with what are presently considered to be the most practical and preferred embodiments, it is to be understood that the invention is not to be limited to the disclosed embodiments, but on the contrary, is intended to cover various modifications and equivalent arrangements included within the spirit and scope of the invention. Also, the various embodiments described above may be implemented in conjunction with other embodiments, e.g., aspects of one embodiment may be combined with aspects of another embodiment to realize yet other embodiments.

Also, the various embodiments described above may be implemented in conjunction with other embodiments, e.g., aspects of one embodiment may be combined with aspects of another embodiment to realize yet other embodiments. In addition, while the invention has particular application to patients who suffer from OSA, it is to be appreciated that patients who suffer from other illnesses (e.g., congestive heart failure, diabetes, morbid obesity, stroke, barriatric surgery, etc.) can derive benefit from the above teachings. Moreover, the above teachings have applicability with patients and non-patients alike in non-medical applications.

What is claimed is:

1. A positive airway pressure (PAP) system, comprising:
a PAP device; and,
a patient circuit, said patient circuit comprising an air delivery conduit and a patient interface unit;
wherein the PAP device is configured to deliver therapeutic treatment pressures based on a reduced calibration thereof, the reduced calibration being substantially independent of the patient circuit.

2. The PAP system of claim 1, further comprising a pressure sensor and/or a flow sensor.

3. A method of delivering therapeutic treatment pressures to a patient via a positive airway pressure (PAP) device comprising an operable flow generator and a patient circuit including a patient interface unit, the method comprising:
generically calibrating the device substantially independent of a specific patient circuit used;
setting a first pressure;
providing a supply of pressurized breathable gas to the patient at or close to the first pressure;
monitoring at least one parameter indicative of a patient's condition over a period of time to measure patient improvement; and,
when the at least one monitored parameter indicates a lack of patient improvement, changing the first pressure.

4. The method of claim 3, further comprising determining a desired motor speed based on the first pressure.

5. The method of claim 3, further comprising controlling the first pressure by controlling a motor speed of the PAP device.

6. The method of claim 3, wherein the actual pressure delivered to the patient is not determined.

7. The method of claim 3, wherein the monitored parameter is flow limitation.

8. The method of claim 7, wherein the at least one monitored parameter indicates the lack of patient improvement by being consistently below a certain threshold.

9. The method of claim 3, wherein the at least one monitored parameter is hourly AHI.

10. The method of claim 9, wherein the at least one monitored parameter indicates the lack of patient improvement by failing to drop below a given value.

11. The method of claim 10, wherein the given value is 2.

12. The method of claim 3, wherein the at least one monitored parameter is an arousal index.

13. The method of claim 12, wherein the at least one monitored parameter indicates the lack of patient improvement by failing to fall.

14. The method of claim 3, wherein the at least one monitored parameter relates to the patient's snore.

15. The method of claim 14, wherein the at least one monitored parameter measures noise during patient expiration.

16. The method of claim 14, further comprising considering the noise during patient expiration as intrinsic device noise.

17. The method of claim 14, wherein the at least one monitored parameter measures noise during patient inspiration.

18. The method of claim 17, further comprising comparing the noise measured during patient expiration and noise measured during patient inspiration.

19. The method of claim 18, further comprising considering the noise measured during patient inspiration in excess of the noise during expiration as patient snore.

20. A system for delivering therapeutic treatment pressure to a patient suffering from sleep disordered breathing, comprising:
a patient circuit operable to deliver the pressurized breathable gas to the patient;
a controllable flow generator operable to generate a supply of pressurized breathable gas to be delivered to the patient at a first pressure substantially independent of the specific patient circuit used;
a monitor operable to measure a parameter indicative of a patient's condition over a period of time; and,
a processor operable to change the controllable flow generator's first pressure when the monitored parameter indicates a lack of patient improvement.

21. The system of claim 20, wherein the monitored parameter is flow limitation.

22. The system of claim 21, wherein the monitored parameter indicates the lack of patient improvement by being consistently below a certain threshold.

23. The system of claim 20, wherein the monitored parameter is hourly AHI.

24. The system of claim 23, wherein the monitored parameter indicates the lack of patient improvement by failing to drop below a given value.

25. The system of claim 24, wherein the given value is 2.

26. The system of claim 20, wherein the monitored parameter is an arousal index.

27. The system of claim 26, wherein the monitored parameter indicates the lack of patient improvement by failing to fall.

28. A method of delivering therapeutic treatment pressure to a patient via a positive airway pressure (PAP) device comprising an operable flow generator and a patient circuit including a patient interface unit, the method comprising:
generically calibrating the device substantially independent of the specific patient circuit used;
setting a first pressure;
providing a supply of pressurized breathable gas to the patient at or near the first pressure;
monitoring a parameter indicative of a patient's condition over a period of time to measure patient improvement; and,
when the monitored parameter indicates a lack of patient improvement changing the first pressure by adjusting an element in the PAP device to modify the amount of pressurized breathable gas provided to the patient.

29. The method of claim 28, wherein the element in the PAP device adjusted to modify the amount of pressurized breathable gas provided to the patient is the motor speed of the PAP device.

30. A system for delivering therapeutic treatment pressure to a patient suffering from sleep disordered breathing, comprising:
a patient circuit operable to deliver the pressurized breathable gas to the patient;
a controllable flow generator operable to generate a supply of pressurized breathable gas to be delivered to the patient at a first pressure substantially independent of the specific patient circuit used;
a monitor operable to measure a parameter indicative of a patient's condition over a Period of time; and,
a processor operable to change the controllable flow generator's first pressure and an element of the controllable flow generator;
wherein the processor changes the first pressure and the element of the controllable flow generator when the monitored parameter indicates a lack of patient improvement.

31. The method of claim 30, wherein the element in the controllable flow generator used to modify the amount of pressurized breathable gas provided to the patient adjusted is the motor speed of the controllable flow generator.

32. A method of classifying mask leak for a patient using a positive airway pressure (PAP) device, the method comprising:
providing a supply of pressurized breathable gas to the patient at a first pressure;
estimating vent flow based on the first pressure;
determining the average value of flow;
determining the mask leak based on the average value of flow and the estimated vent flow; and,
classifying the mask leak according to at least one predetermined mask leak threshold.

33. The method of claim 32, wherein mask leak above the at least one mask leak threshold is classified as high.

34. The method of claim 33, wherein the mask leak below the at least one mask leak threshold is classified as low.

35. The method of claim 33, wherein there is a plurality of predetermined mask leak thresholds.

36. The method of claim 32, further comprising logging the mask leak classification level.

37. A system for treating a patient suffering from sleep disordered breathing, comprising:
a patient circuit configured to deliver pressurized breathable gas to the patient;
a controllable flow generator operable to generate the pressurized breathable gas to be delivered to the patient at a first pressure independent of the specific patient circuit used;
a processor configured to estimate the PAP device's vent flow based on the first pressure, determining the average value of flow, determining the mask leak based on the average value of flow and the estimated vent flow, and classifying the mask leak according to at least one predetermined mask leak threshold; and,
a monitor operable to measure a parameter indicative of a patient's condition over a period of time;
wherein the processor is operable to change the controllable flow generator's first pressure when the monitored parameter indicates a lack of patient improvement.

38. The system of claim 37, wherein the mask leak is classified as high or low.

39. A method of treating a patient via a positive airway pressure (PAP) device, the method comprising:
providing a supply of pressurized breathable gas to the patient at a first pressure;
estimating vent flow based on the first pressure;
determining the average value of flow;
determining the mask leak based on the average value of flow and the estimated vent flow;
classifying the mask leak according to at least one predetermined mask leak threshold;
monitoring at least one parameter indicative of a patient's condition over a period of time to measure patient improvement; and,
when the monitored parameter indicates a lack of patient improvement, changing the PAP device first pressure.

40. A method of treating a patient via a positive airway pressure (PAP) device, the method comprising:
classifying mask leak using the method of claim 32;
monitoring at least one parameter indicative of a patient's condition over a period of time to measure patient improvement; and
changing the PAP device first pressure when the monitored parameter indicates a lack of patient improvement.

41. The system of claim 37, wherein the processor is operable to change the controllable flow generator's first pressure when the monitored parameter indicates a lack of patient improvement and the mask leak is classified below at least one predetermined mask leak threshold.

42. The method of claim 39, wherein the PAP device first pressure is changed when the monitored parameter indicates a lack of patient improvement and the mask leak is classified below at least one predetermined mask leak threshold.

43. The method of claim 40, wherein the PAP device first pressure is changed when the monitored parameter indicates a lack of patient improvement and the mask leak is classified below at least one predetermined mask leak threshold.

44. A method of treating a patient suffering from sleep-disordered breathing, the method comprising:
setting a first pressure;
providing a supply of pressurized breathable gas to the patient at or close to the first pressure via a controllable flow generator;
monitoring a parameter indicative of a patient's condition over a period of time to measure treatment efficacy; and,
when the monitored parameter indicates a change in treatment efficacy, changing the first pressure.

45. The method of claim 44, further comprising adjusting an aggressiveness and/or gentleness associated with the treatment based at least in part on the change in treatment efficacy.

46. The method of claim 44, wherein the monitored parameter is flow limitation.

47. The method of claim 44, wherein the monitored parameter is hourly AHI.

48. The method of claim 44, wherein the monitored parameter is an arousal index.

49. The method of claim 44, wherein the monitored parameter is a patient's snore.

50. The system of claim 1, further comprising an automatic calibration system and/or a learning system.

51. The method of claim 3, further comprising adjusting the supply of pressurized breathable gas based at least in part on an automatic calibration system and/or a learning system.

52. The method of claim 20, further comprising adjusting the supply of pressurized breathable gas based at least in part on an automatic calibration system and/or a learning system.

53. The method of claim 35, further comprising adjusting the supply of pressurized breathable gas based at least in part on an automatic calibration system and/or a learning system.

54. The system of claim 37, further comprising an automatic calibration system and/or a learning system.

55. The system of claim 44, further comprising an automatic calibration system and/or a learning system.

56. The method of claim 46, further comprising adjusting the supply of pressurized breathable gas based at least in part on an automatic calibration system and/or a learning system.

57. The method of claim 51, further comprising adjusting the supply of pressurized breathable gas based at least in part on an automatic calibration system and/or a learning system.
